(19) United States
(12) Patent Application Publication
(10) Pub. No.: US 2013/0238038 A1
(43) Pub. Date: Sep. 12, 2013

(54) ANGLED INFLATABLE COMPOSITE BALLOON AND METHOD

(71) Applicant: Bryan J. Auyoung, Santa Clara, CA (US)

(72) Inventor: Bryan J. Auyoung, Santa Clara, CA (US)

(73) Assignee: KYPHON SARL, Neuchatel (CH)

(21) Appl. No.: 13/414,514

(22) Filed: Mar. 7, 2012

(51) Int. Cl. A61B 17/58; A61M 29/02

(52) U.S. Cl. 606/86 R; 606/192

(57) ABSTRACT

A medical balloon device includes a tube having a longitudinal axis with a distal end portion. Inflatable balloons are coupled longitudinally in series along the distal end portion of the tube. The inflatable balloons are configured to have individually controlled inflation volumes, and the inflatable balloons include different dimensions such that upon inflation of the inflatable balloons a composite profile shape is achieved. Methods of use are disclosed.

### TECHNICAL FIELD

The present disclosure generally relates to medical devices for the treatment of musculoskeletal structures, and more particularly to a surgical system and method employing a balloon configured to present a composite surface that is angled or curved after inflation.

### BACKGROUND

Extremity fractures of a calcaneus or other bone may be reduced percutaneously using Inflatable Bone Tamps (IBTs). While effective, IBTs are typically designed for the spine and the lifting of vertebral bodies. The inflation profiles of these balloons are most effective at lifting flat surfaces. However, calcaneus fractures typically occur on the superior, anterior portion of the bone, which normally has an angled orientation. A single IBT is typically not sufficient to reorient the surface satisfactorily. Many times, multiple balloons are required to return the calcaneus surface to a proper orientation. This disclosure describes an improvement over these prior art technologies.

### SUMMARY

Accordingly, a surgical system and method for correction of a bone injury or disorder are provided. In one embodiment, in accordance with the principles of the present disclosure, a composite balloon system is provided. The system comprises a medical balloon device including a tube having a longitudinal axis with a distal end portion. Inflatable balloons are coupled longitudinally in series along the distal end portion of the tube. The inflatable balloons are configured to have individually controlled inflation volumes, and the inflatable balloons include different dimensions such that upon inflation of the inflatable balloons a composite profile shape is achieved.

In one embodiment, the medical balloon device comprises a tube having a longitudinal axis with a distal end portion. A distal balloon and a proximal balloon are coupled longitudinally in series along the distal end portion of the tube. The distal balloon and the proximal balloon are formed from a compliant material such that a size of each of the balloons is controlled primarily by volume. The distal balloon and the proximal balloon include different dimensions such that upon inflation a composite profile shape is achieved wherein the composite profile includes an angled profile and an angle of the angled profile is increased or decreased based upon the volume in each balloon. The composite profile is configured to engage bone tissue for reducing a fracture.

In one embodiment, a method for repairing a bone is provided. The method comprises the steps of: providing an inflatable bone tamp including a tube having a longitudinal axis with a distal end portion; and a plurality of inflatable balloons coupled longitudinally in series along the distal end portion of the tube, the plurality of inflatable balloons being configured to have individually controlled inflation volumes, the plurality of balloons including different dimensions such that upon inflation of the inflatable balloons a composite profile shape is achieved; providing the bone tamp to a surgical site; and inflating the plurality of inflatable balloons under individual control to form the composite profile shape to reduce a fracture.

### BRIEF DESCRIPTION OF THE DRAWINGS

The present disclosure will become more readily apparent from the specific description accompanied by the following drawings, in which:

FIG. 1 is a side view of one embodiment of components of an inflatable composite balloon system in a deflated state in accordance with the principles of the present disclosure;

FIG. 2 is a side view of the embodiment of FIG. 1 where the inflatable composite balloon system is inflated to a first state for a composite profile in accordance with the principles of the present disclosure;

FIG. 3 is a side view of the embodiment of FIG. 1 where the inflatable composite balloon system is inflated to a second state with a steeper angle for the composite profile in accordance with the principles of the present disclosure;

FIG. 4 is a side view of another embodiment where an inflatable composite balloon system includes three balloons with inflation volumes decreasing distally for a curved composite profile in accordance with the principles of the present disclosure;

FIG. 5 is a side view of another embodiment where an inflatable composite balloon system includes three balloons with inflation volumes increasing distally for a curved composite profile in accordance with the principles of the present disclosure;

FIG. 6 is a schematic side view of another embodiment showing inflation devices coupled to each balloon to provide individual control of each balloon's pressure, fill rate and volume in accordance with the principles of the present disclosure;

FIG. 7 is a schematic view of a calcaneus bone showing a depression/fracture to demonstrate the principles of the present disclosure; and

FIG. 8 is a schematic view of the calcaneus bone of FIG. 7 showing a composite balloon system reducing the depression/fracture in accordance with the present principles.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system and method for bone repair. It is envisioned that the surgical system and method may be employed in applications such as for correction of fractures, depressions and breaks. For example, the surgical system and method can include inflatable bone tamps (IBT) presenting an angled surface for the repair of bones.

In one embodiment, the system and method include an inflatable bone tamp that reduces the complexity of a procedure where a surface for a bone repair needs an angled or curve IBT profile. The IBT has an angled surface provided by employing a series of balloons, which form a composite shape. The balloons may be formed from a compliant material to aid in removing the IBT after use. The composite shape of the IBT provides a sufficient volume to reduce depressions or displaced bone tissues, which is less than conventional IBTs.

In typical procedures, low compliant balloons of larger size, e.g., about 5 cc to about 10 cc may be needed. However, balloons of this size may be difficult withdraw percutaneously without mechanical assistance. To overcome this issue, an IBT is herein provided, which includes multiple balloons mounted in series. Each balloon is controlled so that a controllable angled surface or profile is created. The balloons may be independently controlled or controlled together. For example, a distal balloon may be inflated to a smaller volume than a more proximal balloon to provide an angled surface to move or support bone tissue or the like in performing a repair. In one illustrative example, the distal balloon may have an inflatable volume of, e.g., 1 cc while a proximal balloon would be inflated to 2 cc. The difference in size between the balloons creates the angled surface. The angle could be made steeper by inflating the proximal balloon further while maintaining the volume in the distal balloon. It should be understood that a greater number of balloons may be included and that the shapes and volumes of the balloons may be adjusted or otherwise altered to provide a desired shape.

It is contemplated that one or all of the components of the surgical system may be disposable, peel-pack, pre-packed sterile devices. One or all of the components of the surgical system may be reusable. The surgical system may be configured as a kit with multiple sized and configured components.

It is envisioned that the present disclosure may be employed to treat bones, and in particular extremity bones such as the calcaneus. It should be understood that the present principles are applicable to any bone structures, including but not limited to bones of the spine, legs, feet, arms, etc. It is contemplated that the present disclosure may be employed with other osteal and bone related applications, including those associated with diagnostics and therapeutics. It is further contemplated that the disclosed surgical system and methods may alternatively be employed in a surgical treatment with a patient in a prone or supine position, and/or employ various surgical approaches, including anterior, posterior, posterior mid-line, direct lateral, postero-lateral, antero-lateral, etc. approaches in the calcaneus, spine or other body regions. The present disclosure may also be alternatively employed with procedures for treating the muscles, ligaments, tendons or any other body part. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

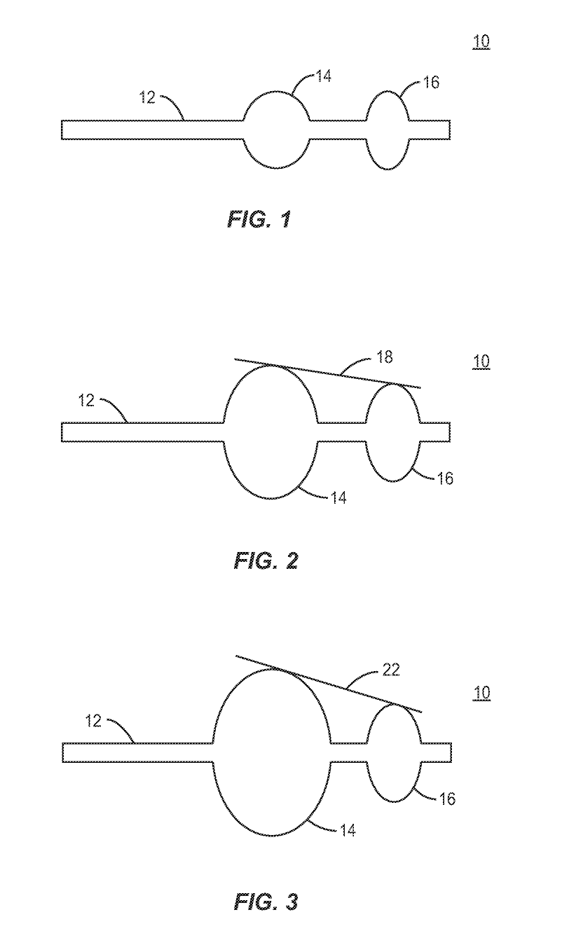

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-8, there are illustrated components of a surgical system, such as, for example, an inflatable balloon system 10 and embodiments in accordance with the principles of the present disclosure.

The components of balloon system 10 can be fabricated from biologically acceptable materials suitable for medical applications, including metals, synthetic polymers, ceramics and bone material and/or their composites, depending on the particular application and/or preference of a medical practitioner. For example, the components of balloon system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of balloon system 10 may have material composites, including the above materials, to achieve various desired characteristics such as strength, rigidity, elasticity, compliance, biomechanical performance, durability and radiolucency or imaging preference. The components of balloon system 10, individually or collectively, may also be fabricated from a heterogeneous material such as a combination of two or more of the above-described materials. The components of correction system 10 may be monolithically formed, integrally connected or include fastening elements and/or instruments, as described herein.

Balloon system 10 is employed, for example, with an open, mini-open or minimally invasive surgical technique to attach move or apply pressure to a bone fragment, fracture or surface, such as, in treating calcaneus fractures. Balloon system 10 includes a flexible longitudinal element or lumen 12, such as, for example, a catheter or other device configured to fluidly communicate with one or both of a proximal balloon 14 and a distal balloon 16. The proximal balloon 14 and the distal balloon 16 may include a compliant membrane that is completely independent from the other balloon or balloons, or a same membrane may be employed that is sealed off from the other balloon or balloons.

Referring to FIG. 1, the proximal balloon 14 and the distal balloon 16 are shown in deflated state. During a procedure, the deflated balloons 14 and 16 are passed through a cannula or other port and delivered to a surgical site. The balloons 14 and 16 are positioned at or near a treatment area, e.g., through bone tissue to a depressed or fractured region. The treatment site may be inside or outside a bone. In useful embodiments, the balloons 14 and 16 are made from a compliant material such as polyurethane or similar materials. The compliant materials make the balloons 14 and 16 easier to withdraw from a sleeve or cannula (not shown) in their deflated state, e.g., after a procedure is performed. As will be understood, employing a composite balloon structure as in system 10, compliant material may be employed since inflation regions can be distributed and of lesser volume to become more effective. The composite structure in accordance with the present principles may include, e.g., a 1 cc balloon 16 and a 2 cc balloon 14 and can perform as well or better than a larger (5 cc-10 cc) non-compliant single balloon.

Referring to FIG. 2, the proximal balloon 14 and the distal balloon 16 are shown in a first inflated state. The proximal balloon 14 and the distal balloon 16 may be inflated separately or together depending on the application and the design of the system 10. The balloons 14 and 16 may be controlled by a single syringe, pump, or other device (not shown) or independently inflated by multiple devices. The inflation device or devices fluidly communicate with the balloons 14 and 16 through lumen 12. There may be one or more paths for fluid communication with balloons 14 and 16 through lumen 12. Lumen 12 may be rigid, semi-rigid or highly flexible.

Multiple balloons 14 and 16 are mounted in series so that by independently controlling the balloons 14 and 16 a controllable composite profile, is this case an angled surface 18 is created. For example, the distal balloon 14 may be inflated to, e.g., 1 cc while the proximal balloon is inflated to, e.g., 2 cc. The difference in size between the balloons 14 and 16 creates the angled surface 18. The angled surface 18 may be employed to contact bone tissue and displace the bone tissue in accordance with the angled surface 18. Other profile shapes, e.g., curve, are also contemplated especially for embodiments having more than two balloons.

It is envisioned that each of the plurality of inflatable balloons employed in system 10 includes a volume of below about 8 cc to reduce the risk of withdrawal difficulties and to ensure better control of the composite profile and individual balloon shapes. In the alternative, the balloons can be pleated so as to provide for a reduced profile when reduced. In addition, a negative pressure can be applied to the balloon once deflated so as to reduce the size of the deflated balloon and aid in the withdrawal of the balloon from the cite of insertion.

Referring to FIG. 3, a proximal balloon 20 and the distal balloon 16 are shown in a second inflated state. The proximal balloon 20 and the distal balloon 16 may be inflated separately or together depending on the application and the design of the system 10. The balloons 20 and 16 may be controlled by a single syringe, pump, or other device (not shown) or independently inflated by multiple devices. The inflation device or devices fluidly communicate with the balloons 20 and 16 through lumen 12.

An angled surface 22 is made steeper by further inflating the proximal balloon 14 to become proximal balloon 20. Proximal balloon 20 may include a volume of, e.g., 3 cc. This additional volume results in a greater displacement and therefore makes the angled surface 22 steeper. The angled surface 22 could be made steeper by inflating the proximal balloon 14 further to become proximal balloon 20 while maintaining the volume in the distal balloon 16. Alternately, the angled surface 22 could be made steeper by deflating the distal balloon 16 while maintaining the volume in the proximal balloon 14 (or balloon 20). A plurality of different inflation and deflation combinations may be implemented to achieve a desired shape and/or displacement using the balloons.

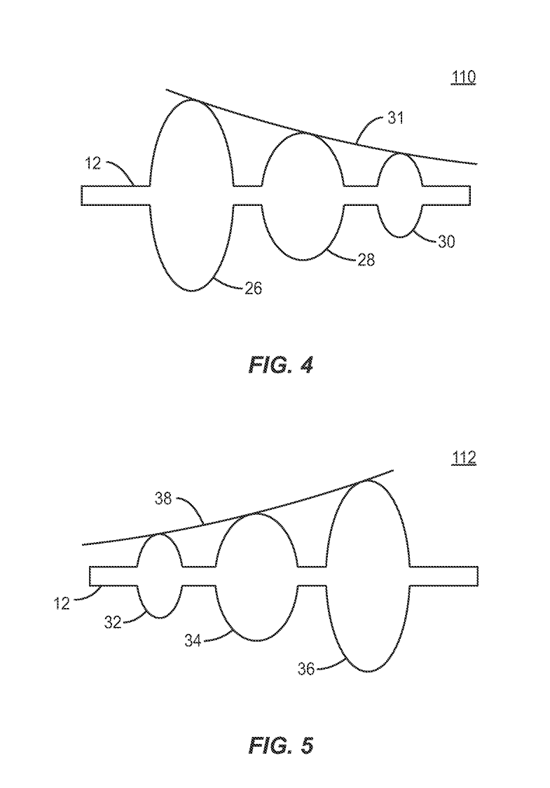

Referring to FIG. 4, a balloon system 110 includes a series of multiple balloons depicted in accordance with another illustrative embodiment. The series of balloons includes three balloons in this case; however, a larger number of balloons may also be employed. The series of balloons includes a proximal balloon 26, middle or central balloon 28 and a distal balloon 30. As before, the balloons 26-30 may be independently controlled by multiple inflation devices (not shown) to provide a profile 31 for applying a force to a fracture, depression or other injury or abnormality in bone or other tissue. In an alternate embodiment, the balloons 26-30 may be inflated and controlled together using a single inflation device (not shown).

Referring to FIG. 5, the balloon system 112 may be reversed to provide an opposite profile 28 using balloons 32-36. The series of balloons includes three balloons in this case; however, a larger number of balloons may also be employed. The balloons 32-36 may include the balloons 26-30 of FIG. 4 inflated to different volumes or may include completely different sized balloons. The series of balloons includes a proximal balloon 32, which is the smallest in this embodiment, a middle or central balloon 34 and a distal balloon 36, which is the largest balloon. As before, the balloons 32-36 may be independently controlled by multiple inflation devices (not shown) to provide the profile 38 for applying a force to a fracture, depression or other injury or abnormality in bone or other tissue. In an alternate embodiment, the balloons 32-36 may be inflated and controlled together using a single inflation device (not shown).

The systems 110 and 112 with a larger number of balloons may be employed if greater control of the profile is needed or if the treatment area is larger. Systems 10, 110 and 112 may be configured to include different spacings between the balloons included therein. In addition, the shapes and sizes of the balloons can be selected to provide a desired result during a procedure. For example, balloons may include shapes such as spheres, cylinders, etc. and have different dimensions to make the balloons narrower or wider in a longitudinal direction, or extend further in a radial direction, etc.

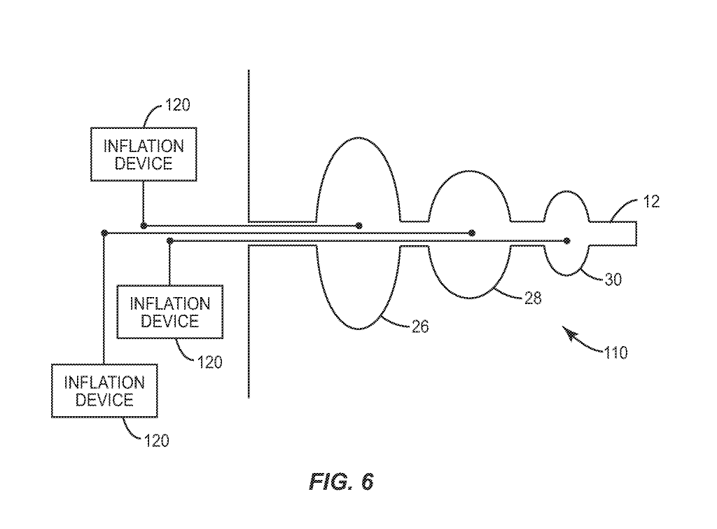

Referring to FIG. 6, a schematic diagram shows the system 110 coupled to a plurality of inflation devices 120. The inflation devices 120 may include syringes, gas pumps, compressed gas cartridges, etc. The inflation devices 120 may include a single gas source with a manifold and independently controlled valves such that the valves may be employed in controlled pressurized fluid flow to the balloons. Other inflation methods are also contemplated. The inflation devices 120 are independently controllable to be able to provide a particular pressure, volume and/or fill rate to each balloon, e.g., balloons 26, 28, 30. The control of the pressurized fluid may be performed manually or automatically.

Automatic control may include the use of a computer interface or controller device (not shown) to set the pressure, volume and/or fill rate automatically based upon a geometric profile desired. For example, one or more of these parameters for each balloon in the desired composite profile, including relative sizes, fill order, pressure/volume and fill speeds, may be controlled and adjusted. In one embodiment, the composite profile may be increased or decreased based upon the individually controlled pressure in each balloon. In another embodiment according to the present disclosure, the composite profile is achieved in part by the use of a compliant material, such as, polyurethane. The individually controlled inflation volumes are controlled, e.g., using a syringe(s) or pump(s) for each balloon. The composite profile is configured to engage bone tissue for reducing a fracture, and the individually controlled inflation volumes may be adjusted in real-time as the fracture is reduced.

In one embodiment in accordance with the present disclosure all or one of the composite balloons may comprise at least two materials that may serve as a reinforcing component and a boundary-forming component. That is, the boundary forming material can be used to achieve a particular angled surface so as to provide the desired overall surface configuration of the device. The boundary-forming component may be any suitable material used for forming a balloon. Examples of such materials are described throughout this disclosure and include materials used in the field. The reinforcing component may provide added tensile strength to the balloon by picking up tensile stress normally applied to the boundary-forming component of the balloon. The reinforcing component may be designed and configured to distribute these forces evenly about its structure, or may be designed and configured to form a space frame for the deployed balloon structure so as to achieve the desired angle and/or shape of the balloons used in the device. The reinforcing component may facilitate better shape control for the balloon, provide for a thinner boundary-forming component, and aid in achieving a repeatable angle once the balloons of the device are inflated.

In one embodiment in accordance with the present disclosure, the reinforcing member component may be a braided matrix extending over selected areas of the balloon. In another embodiment, the braided matrix may enclose the balloon structure in its entirety. In another embodiment, braided matrix is on the inside of the boundary-forming component of the balloon. Conversely, in another embodiment the braided matrix is located on the outside of the boundary-forming component of the balloon. In one embodiment, the braided matrix is located within the boundary-forming component. For example, a boundary-forming component comprising a membrane might include a braided matrix within the membrane. The reinforcing strength of the braided matrix may be influenced by the type of material from which it is constructed, or by the shape and dimension of the individually braided reinforcing members.

Additionally, the reinforcing strength of the braided matrix may be determined by the tightness of the weave. For example, a denser pattern for the braided matrix might provide greater strength but less flexibility, than a less dense weave of a similar pattern. Also, different patterns may have different combinations of physical characteristics. The angle of the intersecting braided members may also be varied to optimize the physical properties of the balloon. The braided matrix may therefore be customized to provide a certain combination of physical or chemical properties. These properties may include tensile and compressive strength, puncture resistance, chemical inertness, shape control, elasticity, flexibility, collapsibility, and the ability to maintain high levels of performance over the long term. The braided materials may be comprised of any suitable material including nitinol, polyethylene, polyurethane, nylon, natural fibers (e.g., cotton), or synthetic fibers.

The boundary-forming component may comprise a synthetic membrane formed from polyurethane or other materials as described for the general balloon construction. The membrane may be coated on the exterior to enhance non-reactive properties between the balloon and the body, to ensure that a balloon will not become bonded to the balloon inflation materials, to lubricate the balloon, and to stiffen the surface to resist puncture. It is expected that a balloon formed from a membrane and braided matrix may designed to operate at an internal pressure of about 300 psi and therefore reduce the possibility of rupture when inflated. As described herein, the size and configuration of the inflation device may vary according to the particular fracture, defect and/or bone to be restored.

Additionally, balloons used in the medical device in accordance with the present disclosure can be single or multi-layered balloons where each balloon layer has the same diameter and/or wall thickness, is comprised of the same material or materials having substantially identical mechanical properties, and has the same degree of molecular orientation in the body portion of the balloon. It will be apparent that in some situations it will be desirable to have some balloon layers having different thicknesses, materials, and/or degree of molecular orientations upon deflation, while at the same time having equivalent size, mechanical properties, and/or orientation upon inflation. For other applications, it will be apparent that one can vary size, material, and/or orientation to at least some degree while still remaining within the spirit of the invention.

In one embodiment of the present disclosure, the balloons of the disclosed medical device comprise an impenetrable structural layer having low friction surfaces so as to facilitate deployment through the delivery tube and prevent rupture of the balloon as it is inflated in situ. It will be apparent that further variations are possible involving different combinations of lubricating layers and structural layers. Structural layers of the balloons of the disclosed medical device can contain polyamides, polyesters, polyethylenes, polyurethanes, their co-polymers and combinations thereof. It will be apparent that further variations are possible involving structural layers of other material or chemical composition.

In one aspect of the embodiments of the present disclosure, the balloons can be adapted to withstand the particular stresses, pressures, and deformities to which they might be placed under when inflated to return the calcaneus surface to a proper orientation. For example, because the top layer might be exposed to sharp objects (such as calcified plaque, bone, bone spurs, or other natural protrusions within a patient's body), the top layer could be made from a more compliant material that is scratch and puncture resistant. In the case of a multi-layer balloon, the outer layer is made from a more compliant material that is scratch and puncture resistant and the inner layers of the multi-layer balloon, which are generally not exposed to sharp objects, made from a less compliant material with a higher burst strength. It will be apparent that further variations are possible, depending on which stresses, pressures, and deformities the layers must withstand in a particular medical application.

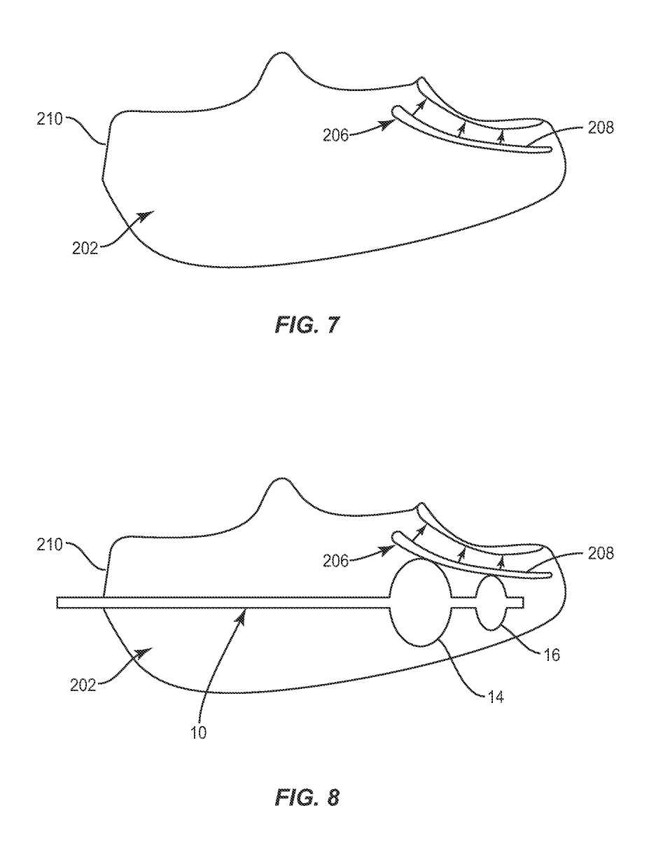

Referring to FIG. 7, a calcaneus bone 202 is illustratively depicted having a depressed region 206 due to a fracture. The depressed region 206 includes a bone fragment 208 that is curved, making conventional inflatable bone tamps difficult to employ to raise the depressed region.

Referring to FIG. 8, the calcaneus bone 202 is illustratively depicted having the depressed region 206 raised using the system 10 embodied as an inflatable bone tamp in accordance with the present principles. The depressed region 206 is accessed percutaneously entering through a heel portion 210 of the calcaneus bone 202. A drill or other tool is employed to open up an access path to the injured portion of the bone 202. A cannula (not shown) is inserted to deploy the system 10. Once system 10 is in place, the balloons 14 and 16 are inflated.

A curvature of the bone fragment 208 is more accurately contacted by an angled profile caused by inflating the two balloons 14 and 16. The angled profile better distributes the support of the depressed region 206 to ensure that the depressed region 206 is raised evenly and more accurately.

The balloons 14 and 16 create the angled profile with compliant material to treat the calcaneus fracture where the multiple balloons give the end user the ability to manipulate the profile of the balloons(s). The present principles provide a more effective method for percutaneously treating calcaneus fractures. The current design will yield better outcomes and reduce procedure time.

In assembly, operation and use, system 10 (and the other systems described above, which will be collectively referred to as system 10 for simplicity) is employed with a surgical procedure, such as, for a correction or treatment of bone fractures. It is contemplated that one or all of the components of system 10 can be delivered or implanted as a pre-assembled device or can be assembled in situ. System 10 may be completely or partially revised, removed or replaced.

For example, as shown in FIGS. 1-8, system 10 (110, 112, etc.), described above, can be employed with a surgical correction treatment of an applicable condition or injury of an affected portion of a, calcaneus bone, bones of the feet or hands, bones of the spine, bones of the arms and legs, etc. and other areas within a body.

In use, to treat a fracture, a medical practitioner obtains access to a surgical site including the fractured bone in any appropriate manner, such as through incision and retraction of tissues. In one embodiment, a drill is employed to remove bone tissue to provide access to a repair site. It is envisioned that system 10 can be used in any existing surgical method or technique including open surgery, mini-open surgery, minimally invasive surgery and percutaneous surgical implantation, whereby the fractured or injured bone is accessed through a mini-incision, or sleeve that provides a protected passageway to the area. Once access to the surgical site is obtained, the particular surgical procedure can be performed for treating the injury or disorder. The configuration and dimension of system 10 is determined according to the configuration, dimension and location of a selected section of the bone fracture and the requirements of a particular application.

An incision is made in the body of a patient and a cutting instrument (not shown) creates a surgical pathway for implantation of components of system 10. This may include the use of a cannula or other device. A preparation instrument (not shown) can be employed to prepare tissue surfaces, as well as for aspiration and irrigation of a surgical region according to the requirements of a particular surgical application.

Other components of system 10 are delivered to the surgical site along the surgical pathway(s). In one embodiment, system 10 includes an agent, which may be disposed, packed or layered within, on or about the components and/or surfaces of system 10. It is envisioned that the agent may include bone growth promoting material, such as, for example, bone graft to enhance fixation of the fixation elements with the bone in need of repair.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines The components of system 10 can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of system 10. Upon completion of the procedure, the surgical instruments and assemblies are removed. The opening drilled in to the bone is filled with a bone cement to provide support for the repaired bone, and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto. The balloon can be modified or extended to accommodate particular formulations of balloon construction materials or fabrication techniques. Different balloon materials and surface coatings, or outer layers of different materials or surface coatings may also be applied to the balloon to facilitate a smaller balloon profile, biocompatibility, lubrication as well as other properties. The embodiments above can also be modified so that some features of one embodiment are used with the features of another embodiment. One skilled in the art may find variations of these preferred embodiments, which, nevertheless, fall within the spirit of the present invention, whose scope is defined by the claims set forth below.

What is claimed is:

1. A medical balloon device, comprising:
a tube having a longitudinal axis with a distal end portion; and
a plurality of inflatable balloons coupled longitudinally in series along the distal end portion of the tube, the plurality of inflatable balloons being configured to have individually controlled inflation volumes, the plurality of inflatable balloons including different dimensions such that upon inflation of the inflatable balloons a composite profile shape is achieved.

2. A medical balloon device as recited in claim 1, wherein the plurality of balloons includes a distal balloon and a proximal balloon wherein the proximal balloon includes a larger inflation volume than the distal balloon so as to provide an angled surface profile when inflated.

3. A medical balloon device as recited in claim 1, wherein the plurality of balloons includes a distal balloon and a proximal balloon wherein the proximal balloon includes a smaller inflation volume than the distal balloon so as to provide an angled surface profile when inflated.

4. A medical balloon device as recited in claim 1, wherein the composite profile includes an angled profile.

5. A medical balloon device as recited in claim 1, wherein the composite profile includes a curved profile.

6. A medical balloon device as recited in claim 1, wherein the plurality of balloons includes a distal balloon, a central and a proximal balloon wherein the proximal balloon includes a larger inflation volume than at least one of the central balloon and the distal balloon so as to provide an angled surface profile when inflated.

7. A medical balloon device as recited in claim 1, wherein the plurality of balloons includes a distal balloon, a central and a proximal balloon wherein the proximal balloon includes a smaller inflation volume than at least one of the central balloon and the distal balloon so as to provide an angled surface profile when inflated.

8. A medical balloon device as recited in claim 1, wherein the plurality of balloons includes a compliant material such that a size of each of the balloons is controlled by an individually controlled pressure.

9. A medical balloon device as recited in claim 8, wherein the composite profile includes an angled profile and an angle of the angled profile is increased or decreased based upon the individually controlled pressure in each balloon.

10. A medical balloon device as recited in claim 8, wherein the compliant material includes polyurethane.

11. A medical balloon device as recited in claim 1, wherein the individually controlled inflation volumes are controlled using a syringe for each balloon.

12. A medical balloon device as recited in claim 1, wherein the individually controlled inflation volumes are controlled using a pump.

13. A medical balloon device as recited in claim 1, wherein the balloon device includes a deflated state, the balloon device being configured to pass through a cannula to be delivered to a surgical site in the deflated state.

14. A medical balloon device as recited in claim 1, wherein the composite profile is configured to engage bone tissue for reducing a fracture.

15. A medical balloon device as recited in claim 14, wherein the individually controlled inflation volumes are adjusted as the fracture is reduced.

16. A medical balloon device as recited in claim 1, wherein the medical balloon device includes an inflatable bone tamp.

17. A medical balloon device as recited in claim 1, wherein the inflated volume of each of the plurality of balloons is less than about 5 cc.

18. A medical balloon device comprising:
a tube having a longitudinal axis with a distal end portion; and
a distal balloon and a proximal balloon, each being coupled longitudinally in series along the distal end portion of the tube, and each being formed from a compliant material such that a size of each of the balloons is controlled by an individually controlled pressure to provide individually controlled inflation volumes,
the distal balloon and the proximal balloon including different dimensions such that upon inflation a composite profile shape is achieved wherein the composite profile includes an angled profile and an angle of the angled profile is increased or decreased based upon the individually controlled pressure in each balloon, the composite profile being configured to engage bone tissue for reducing a fracture.

19. A method for repairing a bone, the method comprising the steps of:
providing an inflatable bone tamp including a tube having a longitudinal axis with a distal end portion; and a plurality of inflatable balloons coupled longitudinally in series along the distal end portion of the tube, the plurality of inflatable balloons being configured to have individually controlled inflation volumes, the plurality of balloons including different dimensions such that upon inflation of the inflatable balloons a composite profile shape is achieved;
providing the bone tamp to a surgical site; and
inflating the plurality of inflatable balloons under individual control to form the composite profile shape to reduce a fracture.

20. A method for repairing a bone as recited in claim 19, wherein inflating the plurality of inflatable balloons includes adjusting the individually controlled inflation volumes as the fracture is reduced.
